(19) United States
(12) Patent Application Publication
(10) Pub. No.: US 2008/0015698 A1
(43) Pub. Date: Jan. 17, 2008

(54) SPINAL DISC IMPLANT

(76) Inventors: James F. Marino, La Jolla, CA (US); David J. Krueger, Cedar Park, TX (US); Erik J. Wagner, Austin, TX (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/774,451

(22) Filed: Jul. 6, 2007

Related U.S. Application Data
(62) Division of application No. 10/698,966, filed on Oct. 31, 2003.
(60) Provisional application No. 60/422,688, filed on Oct. 31, 2002.

(51) Int. Cl. A61F 2/44; A61B 17/58

(52) U.S. Cl. 623/17.15; 606/69; 623/17.16

(57) ABSTRACT

A disc implant is provided which maintains intervertebral spacing and stability of the spine. In an embodiment, a disc implant may include four or more components. Components of the disc implant may include engaging plates and two or more members positioned between the engaging plates. In certain embodiments, a disc implant may include a retainer positioned between one of the engaging plates and one of the members. Complementary portions of the implant components may allow for lateral movement, anteroposterior movement, and/or axial rotation of the engaging members relative to each other during use. In some embodiments, at least one of the members may include a stop to inhibit movement of adjacent vertebrae outside of normal physiological ranges.

### PRIORITY CLAIM

This application claims priority to U.S. Provisional Application No. 60/422,688 entitled “Spinal Disc Implant” filed Oct. 31, 2002. The above-referenced provisional application is incorporated by reference as if fully set forth herein

### BACKGROUND

1. Field of Invention

The present invention generally relates to the field of medical devices, and more particularly to a system for stabilizing a portion of a spinal column. In an embodiment, the system joins together adjacent vertebrae to stabilize a portion of a spine while at least partially restoring range of motion and physiological kinematics.

2. Description of Related Art

An intervertebral disc may be subject to degeneration caused by trauma, disease, and/or aging. A degenerated intervertebral disc may be partially or fully removed from a spinal column. Partial or full removal of the degenerated disc may destabilize the spinal column. Destabilization of the spinal column may reduce a natural spacing between adjacent vertebrae. Reduced spacing between adjacent vertebrae may increase pressure on nerves that pass between vertebral bodies. Increased pressure on nerves that pass between vertebral bodies may cause pain and/or nerve damage.

A disc implant may be inserted into a disc space created by full or partial removal of an intervertebral disc. The disc implant may be inserted using an anterior, lateral, and/or posterior approach. An anterior approach may result in less muscle and tissue damage and/or less bone removal than lateral and/or posterior approaches.

Spinal fusion may involve inserting a disc implant into a space created by full or partial removal of an intervertebral disc. The disc implant may allow and/or promote bone growth between vertebrae to fuse the vertebrae together. The fusion procedure may establish a natural spacing between the adjacent vertebrae and inhibit motion of the vertebrae relative to each other.

A disc implant may be inserted in a space created by full or partial removal of an intervertebral disc. The implant may establish a natural spacing between vertebrae and enhance spinal stability. Intervertebral bone growth may fuse portions of the implant to adjacent vertebrae. The disc implant may allow for movement of adjacent vertebrae relative to each other,

Several patents describe disc implants. U.S. Pat. No. 5,676,701 to Yuan et al., which is incorporated by reference as if fully set forth herein, describes a hard, low-wear, chromium-containing metal ball and socket bearing artificial intervertebral disc that allows unrestricted motion for use in the replacement of spinal disc segments. U.S. Pat. No. 5,401,269 to Buttner-Janz et al., which is incorporated by reference as if fully set forth herein, describes an intervertebral disc endoprosthesis with two prosthesis plates connected to end plates of vertebrae. U.S. Pat. No. 5,314,477 to Marnay, which is incorporated by reference as if fully set forth herein, describes a prosthesis for intervertebral discs designed to replace fibrocartilaginous discs to connect vertebrae of the spinal column.

### SUMMARY

A disc implant may be used to stabilize vertebrae while allowing substantially normal physiological movement of a spine. The disc implant may replace a diseased or defective intervertebral disc. In some embodiments, a disc implant may be assembled from at least four components, including two engaging plates and at least two members positioned between the engaging plates. In some embodiments, a disc implant may include a retainer. The retainer may be positioned between an engaging plate and a member. A disc implant may be positioned between adjacent vertebrae in a spine with each engaging plate contacting a vertebra. Members may be held in position between the engaging plates and relative to each other by natural compression of the spinal column. The members may allow physiological movement of the vertebrae adjacent to the implant. Physiological movement may include axial rotation, axial compression, lateral movement, and/or anteroposterior movement. Anteroposterior movement may allow extension and/or flexion of the spine,

In some embodiments, a disc implant may include two engaging plates and two members. An outer surface of the first engaging plate may couple to a bone surface (e.g., a vertebra). The members may be positioned between the engaging plates. An inner surface of the first engaging plate may have a concave portion. The concave portion of the first engaging plate may complement a first convex portion of the first member. The concave portion of the first engaging plate may promote retention of the first member between the engaging plates. A second convex portion of the first member may complement a concave portion of the second member such that the second member is able to undergo axial rotation, lateral movement, and/or anteroposterior movement relative to the first member. A convex portion of the second member may complement a concave portion of the second engaging plate. The concave portion of the second engaging plate may promote retention of the second member between the engaging plates. An outer surface of the second engaging plate may couple to a second vertebra.

In some embodiments, a disc implant may include two engaging plates, two members, and a retainer. The retainer and the members may be positioned between the engaging plates. An outer surface of each engaging plate may couple to a vertebra. The retainer may complement an inner surface of the first engaging plate. The first member may complement a surface of the retainer. The retainer may promote retention of the first member between the engaging plates during use. A convex portion of the first member may complement a concave portion of the second member to allow axial rotation, lateral movement, and/or anteroposterior movement of the second member relative to the first member. A portion of the second member may complement an inner surface of the second engaging plate. The inner surface of the second engaging plate may promote retention of the second member between the engaging plates.

A disc implant may be used in combination with other devices typically associated with stabilization of a spine. In certain embodiments, a disc implant may be used in combination with spinal fusion procedures.

Members of an implant may be formed from various materials including metals, metal alloys, plastics, ceramics, polymers, and/or composites. Materials may be chosen based on a number of factors including, but not limited to, durability, compatibility with living tissue, and/or surface friction properties. In some implant embodiments, radiological markers may be used in components “invisible” to radiological techniques. In some embodiments, a coefficient of friction an implant component may be adjusted to reduce wear of the component during use.

### BRIEF DESCRIPTION OF THE DRAWINGS

Advantages of the present invention will become apparent to those skilled in the art with the benefit of the following detailed description and upon reference to the accompanying drawings in which:

FIG. 1 depicts an expanded perspective view of components of a disc implant.

FIG. 2 depicts a cross-sectional view of an embodiment of a disc implant.

FIG. 3 depicts a front view of an embodiment of a disc implant.

FIG. 4 depicts an expanded perspective view of an embodiment of a disc implant.

FIG. 5 depicts a cross-sectional view of an embodiment of a disc implant.

FIG. 6 depicts a front view of an embodiment of a disc implant,

FIG. 7 depicts a top view of an engaging plate with one coupling projection.

FIG. 8 depicts a front view of an engaging plate with one coupling projection.

FIG. 9 depicts a top view of an engaging plate with two coupling projections.

FIG. 10 depicts a front view of an engaging plate with two coupling projections.

FIG. 11 depicts a front view of an engaging plate with two coupling projections.

FIG. 12 depicts a top view of an engaging plate with one coupling projection.

FIG. 13 depicts a front view of an engaging plate with one coupling projection.

FIG. 14 depicts a top view of an engaging plate with two coupling projections and a tab with an opening.

FIG. 15 depicts a front view of an engaging plate with two coupling projections and a tab with an opening.

FIG. 16 depicts a top view of an engaging plate with a plurality of coupling projections.

FIG. 17 depicts a front view of and engaging plate with a plurality of coupling projections.

FIG. 18 depicts a cross-sectional view of an embodiment of a member.

While the invention is susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but on the contrary, the intention is to cover all modifications, equivalents, and alternatives falling within the spirit and scope of the present invention as defined by the appended claims.

### DETAILED DESCRIPTION

An intervertebral disc implant may be used to stabilize a portion of a spine. The implant may replace a fibrocartilaginous disc that has degenerated due to natural wear, trauma, or disease. The disc implant may restore a normal separation distance between vertebrae adjacent to the degenerated disc. The implant may allow for normal movement and flexibility of the spine.

Disc implants may allow movement of adjacent vertebrae relative to each other within ranges associated with normal limits for human vertebrae. Disc implants may allow for axial rotation, lateral movement, and/or anteroposterior movement of adjacent vertebrae relative to each other. In a typical human spine, axial rotation may include rotation of about 1°-3° about a longitudinal axis of the spine. An axis of rotation between vertebrae may move in an anterior or posterior direction due to the fibrocartilaginous nature of an intervertebral disc. An axis of rotation between two vertebrae may be located in a posterior direction from a mid-point between the vertebrae. Lateral movement may generally include lateral bending. Lateral bending may include motion to the left and right up to a maximum of about 5° to about 8°. Anteroposterior movement may include flexion and extension. Flexion may include forward (anterior) motion up to a maximum of about 10° to about 15°. Extension may include backward (posterior) motion up to a maximum of about 5° to about 8°.

Embodiments of implants may inhibit movement outside of normal limits for a human spine. Limiting the range of motion of a disc implant during use may decrease chances of injury and allow for normal torso movement. Surrounding tissue and structure adjacent to vertebrae separated by a disc implant may limit some ranges of motion. For example, surrounding tissue and structure may limit axial rotation of vertebrae. Disc implants that allow physiological movement of a spine are described in U.S. Provisional Patent Application Ser. No, 60/422,764 entitled “Movable Disc Implant,” which is incorporated by reference as if fully set forth herein.

In some embodiments, a disc implant may be used to replace a disc in the lumbar region of a spine In some embodiments, a disc implant may be used in the cervical or thoracic regions of a spine. A disc implant may be used independently or in conjunction with other systems or devices to provide stability to the spine. Implantation of a disc implant may be minimally invasive, with only minimal intrusion to adjacent tissue and muscle. A spinal stabilization system may provide minimal risk of dural or neural damage during installation and use.

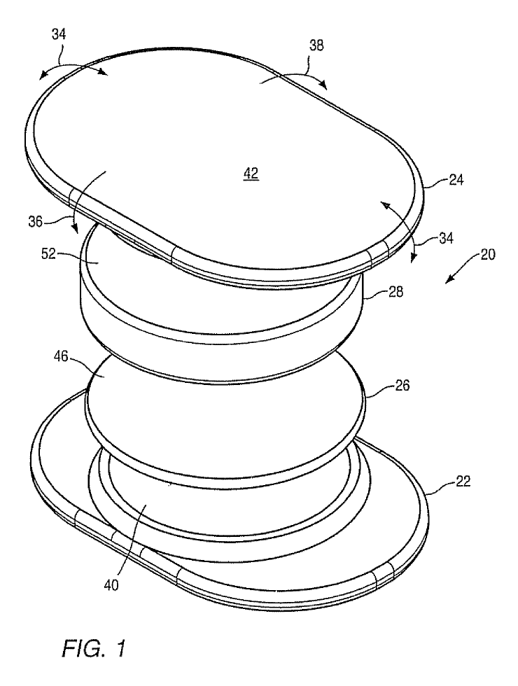

FIGS. 1-3 depict views of an embodiment of a disc implant with four components. FIG. 1 is a perspective view of components of implant 20. Implant 20 may include engaging plates 22, 24 and members 26, 28. In certain embodiments, engaging plates 22, 24 may be substantially identical. Manufacturing costs may be advantageously reduced when engaging plates of a disc implant are substantially the same.

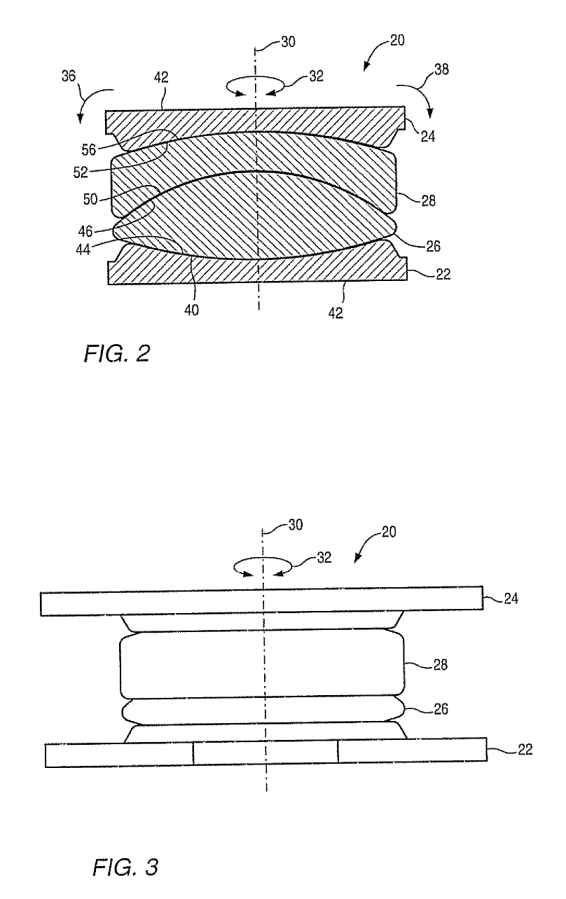

FIG. 1 depicts engaging plate 22 in an inferior position. In some embodiments, engaging plate 22 may assume a superior position. FIG. 2 depicts a cross-sectional side view of assembled implant 20. FIG. 3 depicts a front view of assembled implant 20. Members 26, 28 may be held together as shown in FIGS. 2 and 3 at least in part by pressure resulting from natural spinal compression.

As shown in FIGS. 1 and 2, engaging plate 22 may include inner surface 40 and outer surface 42. Outer surface 42 may be positioned adjacent a bone surface. In an embodiment, outer surface 42 may be coupled to a vertebra. Inner surface 40 may include a concave portion. Surface 44 of member 26 may include a convex portion. Inner surface 40 may complement a portion of surface 44. In some embodiments, inner surface 40 may promote at least partial retention of member 26 between engaging plates 22, 24.

Surface 46 of member 26 may include a convex portion. Surface 50 of member 28 may include a concave portion. Surface 50 may complement at least a portion of surface 46. Surfaces 46, 50 may allow lateral movement, anteroposterior movement, and/or axial rotation of member 28 relative to member 26.

Surface 52 of member 28 may include a convex portion. Inner surface 56 of engaging plate 24 may include a concave portion. Surface 52 may complement at least a portion of inner surface 56. Inner surface 56 may promote at least partial retention of member 28 between engaging plates 22, 24. Engaging plate 24 may include outer surface 42. Outer surface 42 of engaging plate 24 may be positioned adjacent a bone surface. In an embodiment, outer surface 42 may be coupled to a vertebra.

Implant 20 may allow a range of physiological movement of adjacent vertebrae during use. Engaging plate 24 may rotate about axis 30 (as depicted by arrow 32 in FIGS. 2 and 3) relative to engaging plate 22. In some embodiments, member 28 may undergo lateral movement and/or anteroposterior or movement relative to member 26. Movement of member 28 relative to member 26 may allow lateral bending as depicted by arrows 34 in FIG. 1. Movement of member 28 relative to member 26 may also allow flexion and/or extension of engaging plates 22, 24 as depicted by arrows 36 and 38, respectively, in FIGS. 1 and 2.

In some embodiments, a component of implant 20 may translate relative to another component of the implant. For example, member 28 may translate relative to member 26. Relative translation of components of implant 20 may cause axis 30 to shift relative to a center of the implant to allow normal physiological movement of vertebrae adjacent the implant.

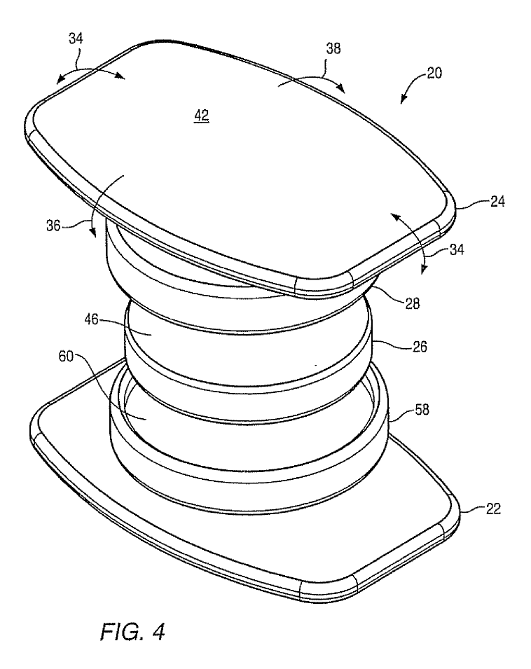

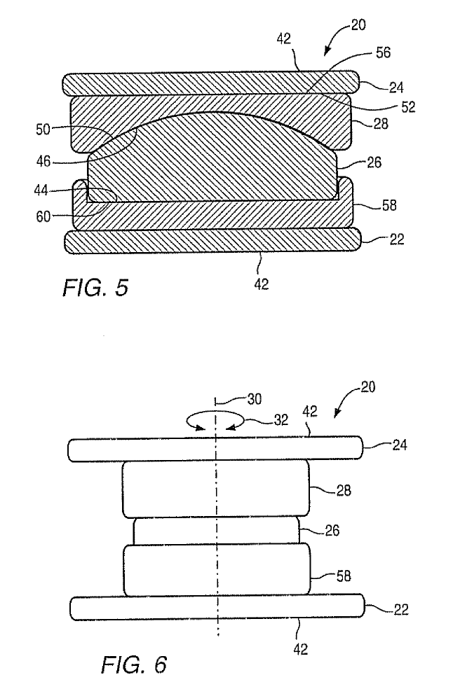

FIGS. 4-6 depict an embodiment of a disc implant with five components. FIG. 4 is a perspective view of components of implant 20. Implant 20 may include engaging plates 22, 24, retainer 58, and members 26, 28. In FIG. 4, engaging plate 22 is shown in an inferior position. In some embodiments, engaging plate 22 may be in a superior position. FIG. 5 depicts a cross-sectional side view of assembled implant 20. FIG. 6 depicts a front view of assembled implant 20. Members 26, 28 may be held together as shown in FIGS. 5 and 6 at least in part by pressure resulting from natural spinal compression.

As shown in FIGS. 5 and 6, engaging plate 22 may include outer surface 42. Outer surface 42 may be positioned adjacent a bone surface. In an embodiment, outer surface 42 may be coupled to a vertebra. An inner surface of engaging plate 22 may complement retainer 58. As shown in FIG. 5, retainer 58 may include a recess. Retainer 58 may promote at least partial retention of member 26 between engaging plates 22, 24. Surface 60 of retainer 58 may complement surface 44 of member 26. Member 26 may rotate relative to retainer 58 about axis 30 as indicated by arrow 32 in FIG. 6.

Surface 46 of member 26 may include a convex portion. Surface 50 of member 28 may include a concave portion. Surface 50 may complement a portion of surface 46 to allow movement of member 28 relative to member 26. Surface 52 of member 28 may complement an inner surface of engaging plate 24. Outer surface 42 of engaging plate 24 may be positioned adjacent a bone surface. In some embodiments, outer surface 42 may be coupled to a vertebra.

Implant 20 may allow a range of physiological movement of adjacent vertebrae during use. Movement of engaging plate 24 relative to engaging plate 22 (i.e., movement of member 28 relative to member 26) may include lateral bending depicted by arrows 34 in FIG. 4 and/or flexion and extension as depicted by arrows 36 and 38, respectively. In certain embodiments, engaging plates 22, 24 may rotate relative to each other about axis 30 (i.e., member 28 may rotate relative to member 26) as indicated by arrow 32 in FIG. 6.

In some embodiments, a component of implant 20 (e.g., member 26, member 28, and/or retainer 58) may translate relative to another component of the implant (e.g., member 26, member 28, retainer 58, and/or engaging plates 22, 24). Relative translation of components of implant 20 may cause axis 30 to shift relative to a center of the implant to allow normal physiological movement of vertebrae adjacent the implant.

In some implant embodiments, components that form the implant may be sized, or include projections or raised surfaces, to limit motion of the implant. For example, a first component of the implant may contact a second component of the implant to limit a maximum amount of flexion to about 15°. In some embodiments, surfaces of components may be configured to contact to limit a maximum extension range, a maximum amount of lateral movement, and/or a maximum amount of axial rotation.

In some embodiments, an outer surface of an engaging plate may include one or more coupling projections to facilitate coupling an implant to a vertebra. In some embodiments, a coupling projection may be formed as a part of an outer surface of an engaging plate. In some embodiments, coupling projections may be affixed to an outer surface of an engaging plate. A coupling projection may be, but is not limited to being, press fit, welded, glued or otherwise coupled to an engaging plate.

Coupling projections on an outer surface of an engaging plate may be inserted into recesses formed in surfaces of vertebrae to inhibit movement of a disc implant relative to the vertebrae and/or provide stability for the implant. In an embodiment, a recess formed in a surface of a vertebra may be a groove. A shape of the groove may complement a shape of a coupling projection.

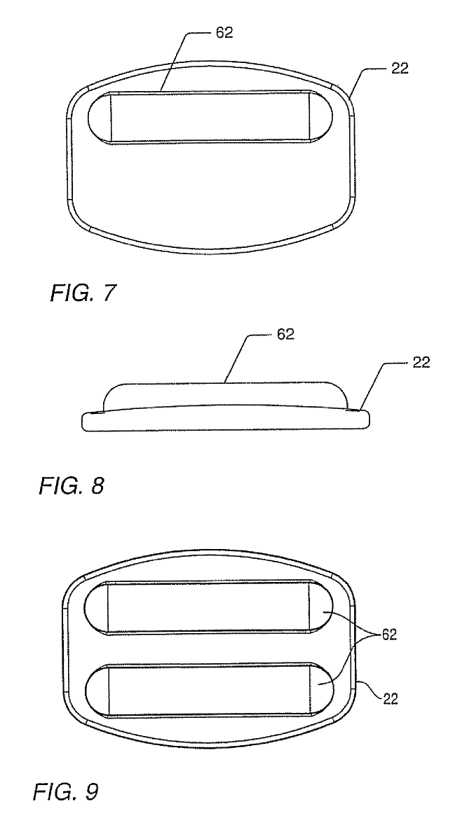

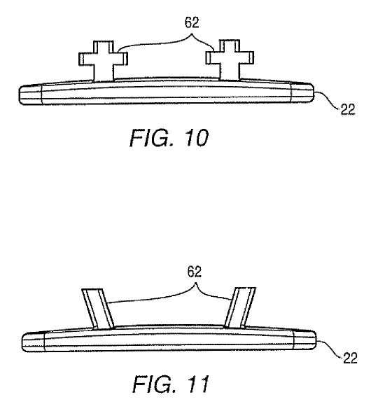

FIGS. 7-17 depict embodiments of coupling projections. As depicted in FIGS. 7-9, coupling projection 62 may have an arcuate shape. A coupling projection with an arcuate shape may be more advantageous than a coupling projection with a shape characterized by sharp angles or corners (e.g., square or rectangular projections). Projections with sharp angles or corners may inhibit distribution of pressure over the surface of coupling projection 62.

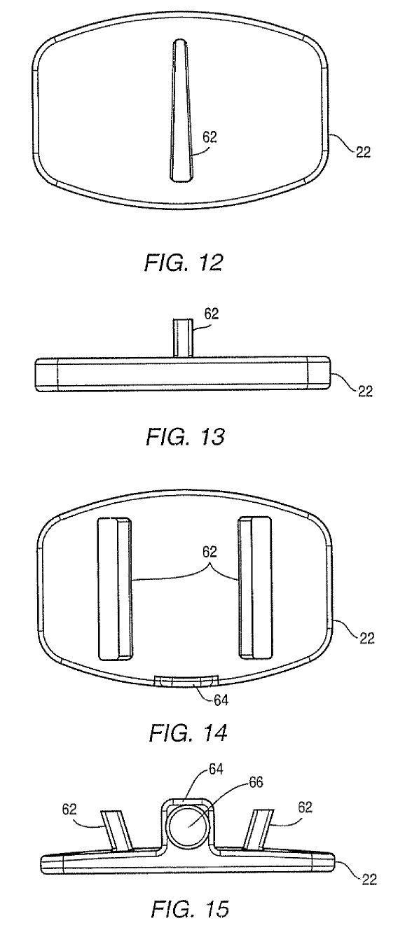

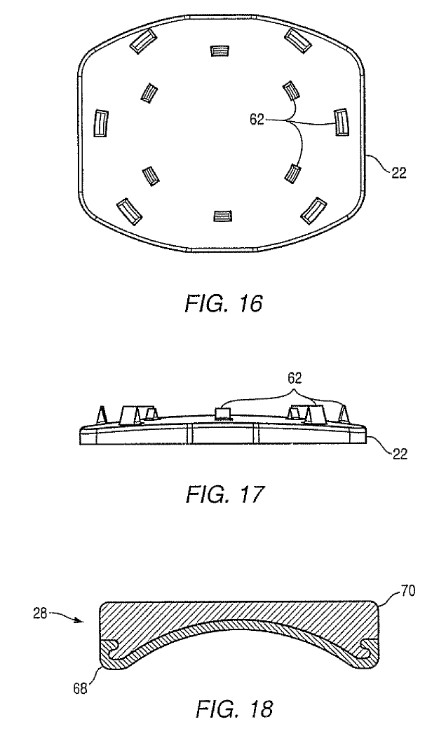

An engaging plate may include one or more coupling projections. FIGS. 7 and 8 depict engaging plates 22 with one coupling projection 62. FIGS. 9-15 depict engaging plates 22 with two coupling projections 62. FIGS. 16 and 17 depict engaging plates 22 with a plurality of coupling projections 62. In some embodiments, coupling projection 62 may have a square, rectangular, trapezoidal, or irregular shape. FIG. 13 depicts coupling projection 62 with a rectangular shape. Coupling projection 62 may be tapered, as shown in FIG 12. Tapered coupling projection 62 may assist in “wedging” the coupling projection into a recess in adjacent bone to form a tight fit. Wedging coupling projection 62 in a recess (e.g., a groove) may inhibit expulsion of engaging plate 22 from an intervertebral space.

A coupling projection embodiment may include spikes or “teeth”. FIGS. 16 and 17 depict an embodiment of coupling projections 62 shaped as spikes. Coupling projections such as those depicted in FIGS. 16 and 17 may “cut” into adjacent bone structures to inhibit movement of engaging plate 22 relative to the adjacent bone structure. In an embodiment, coupling projections of various designs may be used to promote stability of an implant.

In some embodiments, an engaging plate may include one or more openings to allow fastening of the engaging plate to a vertebra. An opening may be positioned on a tab coupled to the engaging plate. In some embodiments, a tab may be an integral part of an engaging plate A fastener may be inserted through an opening in an engaging plate and secured to a vertebra to affix the engaging plate to the vertebra. Fasteners may include, but are not limited to, screws, nails, rivets, trocars, pins, and barbs.

In some embodiments, a fastening system used to couple an engaging plate to a vertebra with a fastener may include a locking mechanism. The locking mechanism may be positioned in an opening of the engaging plate. The fastener may be inserted through the locking mechanism in the opening. After the fastener is secured to the vertebra, the locking mechanism may inhibit backout of the fastener from the vertebra and from the engaging, plate.

In certain embodiments, a locking mechanism may be a ring positioned in an opening in an engaging plate. When the ring is in the opening, a portion of a head of a fastener may contact the ring if the fastener begins to back out of the opening. The ring and fastener head combination may be too large to exit the opening, thus inhibiting backout of the fastener from the vertebra and from the engaging plate. When the ring is inserted into the opening, the ring may lock to a head of the fastener without locking to the engaging plate, thus allowing the engaging plate to be fully tightened down against the vertebra. U.S. Pat. No. 6,454,769 to Wagner et al. and U.S. Pat. No 6,331,179 to Freid et al., both of which are incorporated by reference as if fully set forth herein, describe fastening systems that include a locking mechanism for inhibiting backout of a fastener.

FIGS. 14 and 15 depict an embodiment of tab 64 coupled to engaging plate 22. Tab 64 may include openings 66. During installation, engaging plate 22 may be positioned such that tab 64 abuts an adjacent bone structure. A fastener may be inserted through opening 66 and directly into the adjacent bone structure (forming an opening) or into a pre-formed opening in the bone. In some embodiments, a locking mechanism may be coupled to a fastener before insertion of the fastener in an opening in an engaging plate. In certain embodiments, a locking mechanism may be positioned in an opening of an engaging plate before insertion of a fastener into the opening. Once secured, the fastener and the locking mechanism may inhibit movement of engaging plate 22 relative to an adjacent bone structure.

In some disc implant embodiments, one or more implant components may be curved to correspond to a lordotic curve of a spine. Several different implants with differing lordotic angle may be provided to a surgeon who will install a disc implant in a patient. The surgeon may choose a disc implant that will provide desired lordosis for the patient. Lordotic indications may be etched or otherwise marked (e.g., color coded) on a portion of a disc implant to indicate the amount of lordosis provided by the implant. In an embodiment, a cervical disc implant may have about 5°-20° (e.g., about 12°) of curvature to accommodate lordosis.

In some embodiments, an implant may be curved to accommodate radial curvature of vertebrae. Implants may be provided with varying amounts of radial curvature. Disc implants may be provided in large, medium, and small radial curvature sizes. An indication of an amount of radial curvature provided by an implant may be etched or otherwise marked on a portion of the implant.

Implant components may be made of biocompatible materials including, but not limited to, metals, alloys, ceramics, polymers, and/or composites. For example, an alloy may include cobalt-chrome-molybdenum (CoCrMo). Ceramics may include, but are not limited to, alumina, zirconia, or composites. Polymers used for implant components may include ultra-high molecular weight polyethylene, polyfluorocarbons, and/or polyesteresterketone (PEEK). In some embodiments, implant components may be formed of titanium, titanium alloys, steel, and/or steel alloys. In addition, materials may be chosen based upon characteristics such as durability and ease with which biological tissue, such as human bone, fuses with the material. For example, titanium typically fuses well with bone but may wear poorly over time. A cobalt-chrome-molybdenum alloy may wear well, but may not fuse as well with biological tissue.

In certain embodiments, implant components may be formed of different materials. For example, adjacent components may be formed of different materials to minimize wear of the components over time. In an embodiment, engaging plates and/or a retainer may be formed from titanium or cobalt-chromali and members may be formed from ceramic (e.g., alumina), polymer (e.g., ultra-high molecular weight polyethylene), or combinations thereof.

In some embodiments, engaging plates and/or members may be or may include bioabsorbable material. Surfaces of engaging plates and/or members that contact bone may include a coating to promote osseointegration of the implant with bone. The coating may be, but is not limited to, a bone morphogenic protein, hydroxyapatite, and/or a titanium plasma spray.

In an embodiment, an implant component may be formed from two or more materials. FIG. 18 depicts layers 68, 70 of member 28. Layer 68 may include metal or alloy (e.g., cobalt-chromali). Layer 70 may include polymer (e.g., ultra-high molecular weight polyethylene). Layers 68 and 70 may be molded together. In some embodiments, complementary shapes (e.g., mating surfaces) of layers 68 and 70 may couple the layers together.

In certain embodiments, an implant may be distributed and/or sold pre-assembled and stored in sterile packaging until needed. In some embodiments, one or more implant components may include radiological markers. Markers may be coupled to or incorporated into materials that are “invisible” to X-rays (e.g., polymers). The ability to “see” all of the members of a disc implant would allow a surgeon to determine a location and/or relative alignment of members without invasive procedures.

In some embodiments, a contact surface of a component may be treated to adjust the coefficient of friction of the contact surface so that the component has desired movement relative to an adjacent component. A contact surface of a component may be machined, formed, and/or chemically treated to establish a desired coefficient of friction. The desired coefficient of friction may allow for reduction of wear of the component. In some implant embodiments, an insert, coating, liner, or other covering may be placed on all or part of a contact surface of a component. The insert, coating, liner, or covering may modify frictional or other physical properties of the component relative to another component.

To insert an artificial disc, a surgical opening may be formed in a patient to allow access to an intervertebral disc that is to be replaced. A discectomy may be performed to remove the intervertebral disc or a portion of the intervertebral disc. Trials may be used to establish a spacing between vertebrae. The trials may be used to determine the height of an artificial disc that is to be inserted into the disc space formed by the discectomy.

If the artificial disc has coupling projections, such as the coupling projections depicted in FIGS. 7-15, a chisel guide plate may be inserted in the disc space. The chisel guide plate may be used in conjunction with a drill and/or chisel to form appropriate openings for coupling projections in vertebrae that the artificial disc is to be positioned between.

The vertebrae may be distracted a sufficient distance to allow for insertion of the artificial disc. The artificial disc may be inserted into the disc space, and distraction may be removed. In some embodiments, a binder may be used to hold the artificial disc together during insertion of the artificial disc into the disc space. After insertion, the binder may be removed. In some embodiments, an insertion instrument may hold the artificial disc together during insertion of the artificial disc into the disc space.

In this patent, certain U.S. patents, U.S. patent applications, and/or U.S. provisional patent applications have been incorporated by reference. The text of such patents and applications, are, however, only incorporated by reference to the extent that no conflict exists between such text and the other statements and drawings set forth herein. In the event of such conflict, then any such conflicting text in such incorporated by reference U.S. patents and U.S. patent applications is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1.-13. (canceled)

14. A disc implant comprising:
engaging plates;
a retainer positioned adjacent one of the engaging plates during use;
two members positionable between the retainer and one of the engaging plates; and
wherein relative motion of the members allows lateral movement, anteroposterior movement, and axial rotation of the engaging members relative to each other during use.

15. The implant of claim 14, wherein a surface of the retainer comprises a recess, wherein one of the members comprises a surface complementary to at least a portion of the recess, and wherein the complementary surface of the member is positionable in the recess.

16. The implant of claim 14, wherein at least one of the members comprises a stop configurable to limit movement of the engaging plates relative to each other during use.

17. The implant of claim 14, wherein the retainer comprises a stop configurable to limit movement of the engaging plates relative to each other during use.

18. The implant of claim 14, wherein at least one of the engaging plates comprises a stop configurable to limit movement of the engaging plates relative to each other during use.

19. The implant of claim 14, wherein at least one of the engaging plates comprises at least one coupling projection.

20. The implant of claim 14, wherein the retainer comprises a substantially circular shape.

21. A spinal implant for placement within a disc space, said spinal implant comprising:
a first engaging plate, said first engaging plate comprising an outer surface and an inner surface;
a second engaging plate, said second engaging plate comprising an outer surface and an inner surface;
a first member, said first member comprising a first surface and a concave surface;
a second member, said second member comprising a first surface and a convex surface;
a retainer, said retainer comprising a first surface and a second surface;
wherein the outer surface of the first engaging plate is configured to be coupled to a first vertebra;
wherein the inner surface of the first engaging plate is coupled to the first surface of the first member;
wherein the concave surface of the first member is coupled to the convex surface of the second member;
wherein the first surface of the second member is coupled to the first surface of the retainer;
wherein the second surface of the retainer is coupled to the inner surface of the second engaging plate, and
wherein the outer surface of the second engaging plate is configured to be coupled to a second vertebra.

22. The spinal implant of claim 21, wherein the first engaging plate comprises titanium.

23. The spinal implant of claim 21, wherein the first member comprises polyesteresterketone, and
wherein the second member comprises polyesteresterketone.

24. The spinal implant of claim 21, wherein the retainer comprises a recess, said recess and retainer promoting at least partial retention of second member between first engaging plate and second engaging plate.

25. The spinal implant of claim 21, wherein the outer surface of the first engaging plate comprises a coupling projection,
wherein said coupling projection is configured to be wedged into a groove of the first vertebra to inhibit expulsion of first engaging plate from the disc space.

26. The spinal implant of claim 25, wherein the coupling projection is a spike.

27. The spinal implant of claim 21, wherein the outer surface of the first engaging plate comprises a coating, and
wherein the coating promotes osseointegration.

28. The spinal implant of claim 21, further comprising a removable binder,
wherein said binder is configured to hold the first engaging plate, the first member, the second member, the retainer, and the second engaging plate together during insertion of the spinal implant within the disc space, and
wherein after insertion, said binder is configured to be removed from the spinal implant.

29. A spinal implant for placement within a disc space, said spinal implant comprising:
a first engaging plate, said first engaging plate comprising an outer surface and an inner surface;
a second engaging plate, said second engaging plate comprising an outer surface and an inner surface;
a first member, said first member comprising a first surface and a convex surface;
a second member, said second member comprising a first surface and a concave surface;
a retainer, said retainer comprising a first surface and a second surface;
wherein the outer surface of the first engaging plate is configured to be coupled to a first vertebra;
wherein the inner surface of the first engaging plate is coupled to the first surface of the first member;
wherein the convex surface of the first member is coupled to the concave surface of the second member;
wherein the first surface of the second member is coupled to the first surface of the retainer;
wherein the second surface of the retainer is coupled to the inner surface of the second engaging plate, and
wherein the outer surface of the second engaging plate is configured to be coupled to a second vertebra.
